(19) United States
(12) Patent Application Publication
(10) Pub. No.: US 2022/0031178 A1
(43) Pub. Date: Feb. 3, 2022

(54) CONNECTION SYSTEM FOR A SPHYGMOMANOMETER

(71) Applicant: SPENGLER SAS, Aix-en-Provence (FR)

(72) Inventors: Nicolas BRULET, Peyrolles en Provence (FR); Patrick JOUFFRET, Toulon (FR)

(73) Assignee: SPENGLER SAS, Aix-en-Provence (FR)

(21) Appl. No.: 17/278,474

(22) PCT Filed: Sep. 26, 2019
(86) PCT No.: PCT/FR2019/052290

§ 371 Date: Mar. 22, 2021

(30) Foreign Application Priority Data
Sep. 26, 2018 (FR) 1858820

(51) Int. Cl. A61B 5/022; A61B 5/021

(52) U.S. Cl. CPC A61B 5/02233; A61B 2562/225; A61B 5/02141

(57) ABSTRACT

A connection system for a sphygmomanometer including: an inflatable cuff adapted to be placed on a limb of a patient, the cuff includes an inflatable pouch, a flexible tube including a first end and a second end, the first end in fluid communication with the inflatable pouch of the cuff, an inflation bulb formed by an inflator bulb connected to a pressure gauge for measuring blood pressure, a connection member mounted in the inflation bulb to connect the second end of the tube to the inflation bulb, the tube adapted to provide a direct link between the inflation bulb and the cuff, the second end of the tube includes a male end-piece and the connection member includes a female connector for removably receiving the male end-piece, the connection member includes a portion that cooperates with a complementary portion provided on the male end-piece of the tube.

### TECHNICAL FIELD OF THE INVENTION

The object of the invention is a connection system for a sphygmomanometer.

It relates to the technical field of medical devices, and more particularly that of devices that make it possible to measure blood pressure.

### PRIOR ART

The measurement of blood pressure is one of the most common measurements taken by doctors. A quick and precise measurement is therefore beneficial whether for the doctor or for the patient. The preferred method for taking such a measurement remains the use of a tensiometer (or sphygmomanometer).

In reference to FIG. 1, this measurement of the blood pressure is generally taken using:

an inflatable cuff 1 adapted to be placed around the limb (here the arm) of a patient to be examined and/or monitored, which cuff includes an inflatable pouch,

a measurement system, such as a pressure gauge 2 for reading the blood pressure that is connected to the cuff 1,

an inflator bulb 3, itself connected to the cuff 1 by a flexible tube 4, used to increase the air pressure in said cuff. This bulb 3 is generally provided with a valve of which the control device 30 makes it possible to control the pressure and to make it decrease progressively in order to take the measurement.

Different types of sphygmomanometers have been developed, including the inflation bulb MP shown in FIG. 1. The pressure gauge 5 and the inflator bulb 3 are joined together. The flexible tube 4 connects the inflation bulb MP to the cuff 1 to provide the inflation of the latter.

The cuff 1 has to be positioned around the limb, in such a way that its inflatable pouch is positioned at an artery. The pouch is inflated to first bring the pressure of the cuff 1 to a value greater than the systolic pressure in order to block the arterial circulation in the limb. By adjusting the control device 30, the pressure of the cuff 1 is then progressively decreased to the limit value from which the blood pressure is sufficient to again allow the blood to pass in the artery. This is the systolic pressure. By continuing with the deflating, the pressure of the cuff 1 is brought to a value from which there is no longer any obstacle to the arterial flow even when the heart is in diastole. This is the diastolic pressure.

There are many causes that can lead to an incorrect measurement of blood pressure. The choice of the sphygmomanometer used is therefore paramount for the diagnostic not to be erroneous.

The ergonomics of an inflation bulb MP and/or the display system of the pressure gauge 5 (needle dial, digital display, etc.) can change from one brand to another or according to the models of inflation bulbs. Also, the dimensions of the cuff 1 must be proportioned to the morphology of the limb of the patient, and in particular to its circumference. A cuff that is excessively narrow will overestimate the blood pressure and a cuff that is excessively wide will underestimate it. In these two cases, the value of the blood pressure obtained is not representative and does not make it possible to carry out a correct diagnosis. The choice of the size of the cuff used is therefore paramount for the diagnosis not to be erroneous. Therefore, the fact that a doctor can easily change the inflation bulb and/or cuff as needed appears advantageous.

A connection system for a sphygmomanometer is known in patent document FR3051347 (SPENGLER), comprising:

an inflatable cuff,

a flexible tube including a first end and a second end, the first end being in fluid communication with the inflatable pouch of the cuff,

an inflation bulb,

a connection member mounted in the inflation bulb to connect the second end of the flexible tube to said inflation bulb,

the second end of the tube includes a male end-piece and the connection member includes a female connection hole for removably receiving said male end-piece,

the male end-piece includes several O-ring seals spaced longitudinally, which seals cooperate with the inner wall of the female connection hole to form an airtight seal.

Such a connection system makes it possible to have a sphygmomanometer with an interchangeable configuration. The doctor can now very easily change the inflation bulb while still using the same cuff, or inversely to adapt to the morphology of the patient. However, in practice, the connection of the male end-piece in the female connection hole can be relatively long. In addition, a disconnection of the cable following an untimely pulling off of the tube cannot be excluded. This connection can furthermore be difficult due to the presence of the O-ring seals on the male end-piece and due to the friction that they generate. Furthermore, these seals can be degraded when the male end-piece is not connected in the female connection hole, and left open to the outside environment, which can, over time, give rise to air-tightness problems.

An objective of the invention is to propose a connection system for a sphygmomanometer that facilitates the assembly between the connection member of the inflation bulb and the second end of the flexible tube, in such a way as to change the inflation bulb and/or cuff even more quickly.

Another objective of the invention is to propose a connection system for a sphygmomanometer of which the design is particularly robust.

Yet another objective of the invention is to propose a connection system for a sphygmomanometer of which the design is simple, inexpensive and that is simple to use.

### DISCLOSURE OF THE INVENTION

The solution proposed by the invention is a connection system for a sphygmomanometer comprising:

an inflatable cuff adapted to be placed on a limb of a patient to be examined and/or monitored, which cuff includes an inflatable pouch,

a flexible tube including a first end and a second end, the first end being in fluid communication with the inflatable pouch of the cuff,

an inflation bulb formed by an inflator bulb connected to a pressure gauge for measuring blood pressure,

a connection member mounted in the inflation bulb to connect the second end of the tube to said inflation bulb, such that said tube is adapted to provide a direct link between said inflation bulb and the cuff,

the second end of the tube includes a male end-piece and the connection member includes a female connection hole for removably receiving said male end-piece.

This system is noteworthy in that:

the connection member comprises a portion that cooperates magnetically with a complementary portion provided on the male end-piece of the tube,

at least one of the portions includes a permanent magnet providing magnetization.

The magnetic connection between the tube and the inflation bulb offers a simple and robust solution that makes it possible to very simply and very quickly change the inflation bulb and/or cuff. Furthermore, the supply force makes it possible to reduce the risks of untimely disconnection of the cable.

Other advantageous characteristics of the invention are listed hereinbelow. Each one of these characteristics can be considered individually or in combination with the noteworthy characteristics defined hereinabove, and be the object, where applicable, of one or more divisional patent applications:

According to an embodiment, a junction block is mounted in the inflation bulb, which junction block is adapted to provide a fluidic communication between the inflator bulb, the pressure gauge, a device for controlling a valve, and the connection member. The connection member is an added interface part inserted into an end of the junction block.

According to an alternative embodiment, a junction block is mounted in the inflation bulb, which junction block is adapted to provide a fluidic communication between the inflator bulb, the pressure gauge, a device for controlling a valve, and the connection member. The connection member is an integral part of the junction block and forms with the latter a single-piece part.

Advantageously, the portion of the connection member is in the form of a collar.

Advantageously, the complementary portion provided on the male end-piece of the tube is in the form of a collar, which collar is disposed outside of said tube and bears against the second end of said tube.

The collar of the male end-piece advantageously bears against the collar of the connection member, when said male end-piece is connected in the female hole of said connection member.

According to an embodiment, the portion of the connection member includes a permanent magnet. And the complementary portion provided on the male end-piece includes a ferromagnetic material.

According to an alternative embodiment, the portion of the connection member includes a ferromagnetic material. And the complementary portion provided on the male end-piece includes a permanent magnet.

According to another alternative embodiment, the portion of the connection member and the complementary portion provided on the male end-piece each include a permanent magnet, said permanent magnets being arranged in such a way that their poles of opposite nature are facing each other and attract each other.

Advantageously, the complementary portion provided on the male end-piece is an added part integral with said male end-piece.

According to an alternative embodiment, the complementary portion provided on the male end-piece is an integral part of said male end-piece and forms with the latter a single-piece part.

According to an embodiment, a junction block is mounted in the inflation bulb, which junction block is adapted to provide a fluidic communication between the inflator bulb, the pressure gauge, a device for controlling a valve, and the connection member. This junction block is provided with a seal that cooperates with the male end-piece of the tube to provide an airtight seal between said male element and said junction block.

According to an alternative embodiment, the female connection hole of the connection member is provided with a seal that cooperates with the male end-piece of the tube to provide an airtight seal between said male element and said female hole.

Advantageously, the male end-piece of the tube has an outer wall devoid of an air sealing element.

Advantageously, a connection member is mounted in the cuff to connect the first end of the tube to said cuff. The first end of the tube includes a male end-piece and the connection member of the cuff includes a female connection hole for removably receiving said male end-piece. The connection member of the cuff comprises a portion that cooperates magnetically with a complementary portion provided on the male end-piece of the first end of the tube. At least one of said portions includes a permanent magnet providing magnetization.

### DESCRIPTION OF THE FIGURES

Other advantages and characteristics of the invention shall appear better when reading the following description of a preferred embodiment, in reference to the accompanying drawings, given as indicative and non-limiting examples and wherein:

FIG. 1 is a diagrammatical representation of a system according to the invention where the cuff is installed on the arm of a patient,

FIG. 2 is a perspective view showing the connection of the second end of the tube on an inflation bulb,

FIG. 3 is a diagrammatical vertical cross-section view of a sphygmomanometer according to the invention,

FIG. 4 is a cross-section representation of a male end-piece of the tube and of the connection member mounted in the inflation bulb,

FIG. 5 shows, in a disassembled manner, the connection elements of FIG. 4,

FIG. 6a is a cross-section representation of a male end-piece of the tube and of the connection member mounted in the inflation bulb, in an embodiment alternative,

FIG. 6b is a cross-section representation of a male end-piece of the tube and of the connection member mounted in the inflation bulb, in another embodiment alternative,

FIG. 7 is a diagrammatical representation of the two ends of a tube according to the invention,

FIG. 8 is a diagrammatical representation of an embodiment alternative of a system according to the invention where the cuff is installed on the arm of a patient,

FIG. 9 is a perspective view showing the connection of the first end of the tube on a cuff.

### PREFERRED EMBODIMENTS OF THE INVENTION

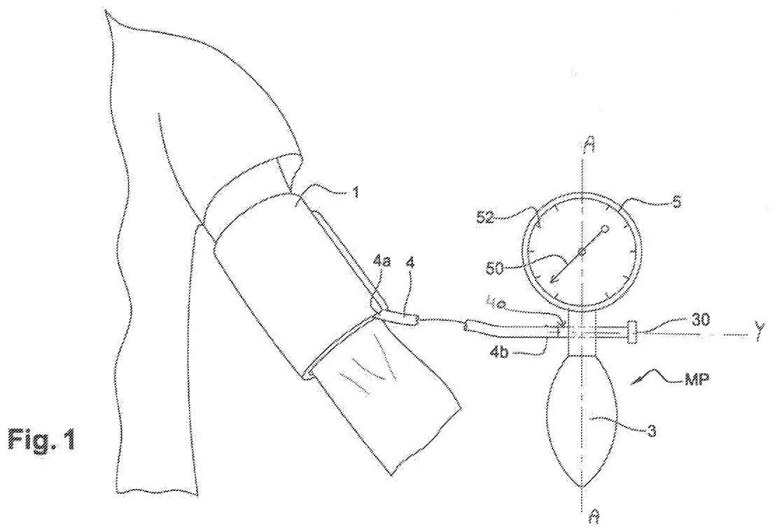

In reference to FIG. 1, the system object of the invention includes the following elements:

an inflatable cuff 1 adapted to be placed on the arm of a patient to be examined and/or monitored, which cuff includes an inflatable pouch,

an inflating means 3, such as an inflator bulb, connected to a flexible tube 4 in order to provide the inflation of the cuff 1,

a pressure gauge 5 for measuring the systolic and diastolic blood pressure of the patient, which pressure gauge is connected to the inflator bulb 3 to form an inflation bulb MP.

In FIG. 1, the cuff 1 is installed around the arm of a patient, at the artery, in such a way as to take the various blood pressure measurements. The flexible tube 4 makes it possible to bring air for the inflation of the cuff 1. When it is inflated, the cuff 1 crushes the artery on which it is placed. By listening, using a stethoscope, the noise emitted by the blood during the passage thereof in the artery, two values of blood pressure are measured. Thus, when the cuff 1 is sufficiently inflated to compress the artery, the blood cannot no longer pass and the doctor does not perceive the noise. The cuff 1 is then deflated progressively, making it possible to perceive the noise of the pulse beats defining the systolic blood pressure (Maximum pressure). This noise is amplified than disappears, the pressure measured then corresponding to the diastolic blood pressure (Minimum pressure).

The cuff 1 can be of different sizes so as to be able to be adapted to patients with different morphologies and arms diameters. It has for example a length comprised between 40 cm and 100 cm, a width that can vary between 5 cm and 30 cm. The cuff 1 is made in a conventional manner and includes an inflatable pouch. It can be manufactured in a single piece, with a double wall delimiting the inflatable pouch, and be made from a waterproof flexible plastic material such as a polymer or an elastomer, such as polyethylene or polyurethane. Preferably, it is made from a thermoplastic material. The cuff 1 can also be formed by two walls made of non-waterproof textile, such as cotton, which walls delimit a pouch wherein the inflatable pouch is housed, which inflatable pouch is made from a waterproof flexible plastic material such as a polymer or an elastomer.

An inflating means 3 makes it possible to inject air into the inflatable pouch of the cuff 1 to inflate it. In FIG. 1, this inflating means 3 preferably is in the form of an inflator bulb, which bulb is of the type usually used in the sphygmomanometer systems known from prior art. It is sufficient for the doctor to exert, manually, successive presses on the bulb 3 to inject air into the tube 4 and inflate the cuff 1. The bulb 3 is connected to a member 30 for controlling a control valve or a valve for venting the inflatable pouch of the cuff 1. Actuating this member 30 makes it possible in particular to gradually release the pressure of the inflated cuff 1 for the purpose of determining systolic pressures. This member 30 can be in the form of a wheel or a push-button.

The pressure gauge 5 shown in FIG. 1 has a needle 50 that cooperates with a graduation 52 on a reading dial, to indicate the blood pressure. The pressure gauge 5 can however be provided with a digital display screen.

The tube 4 is made from a flexible material, such as a polyethylene, a polystyrene, or from any other plastic material that suits those skilled in the art. It has a diameter that can vary from 0.2 cm to 2 cm, and a length comprised between 20 cm and 2 m. It includes a first end 4a and a second end 4b. The first end 4a is in fluidic communication with the inflatable pouch of the cuff 1. This first end 4a can be directly welded on the inflatable pouch of the cuff 1, without possibility of disconnection.

FIG. 1 diagrams a configuration where the pressure gauge 5 is connected to the inflator bulb 3 to form an inflation bulb MP. This unique device MP is known to those skilled in the art. The inflation bulb MP is connected to the cuff 1 using the tube 4 allowing air to circulate from the bulb 3 to the inside of the inflatable pouch of said cuff.

The system includes removable means of connection constituted of arrangements in the form of male end-pieces and female holes, of the quick assembly type.

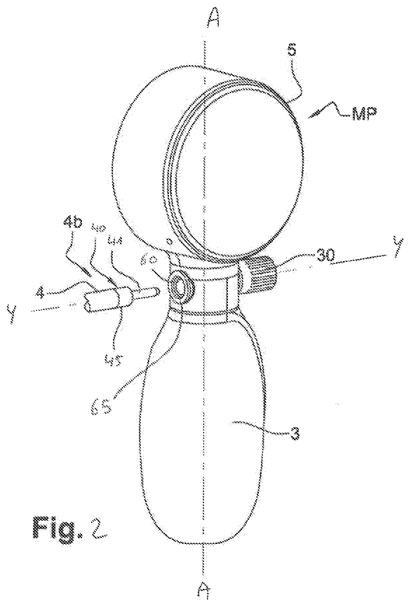

In FIGS. 2 to 7, the tube 4 includes means of connection 40 located on at least the second end 4b and preferably on each of its ends 4a and 4b. These means of connection 40 make it possible in particular to connect the tube 4 to the inflation bulb MP and optionally to the cuff 1.

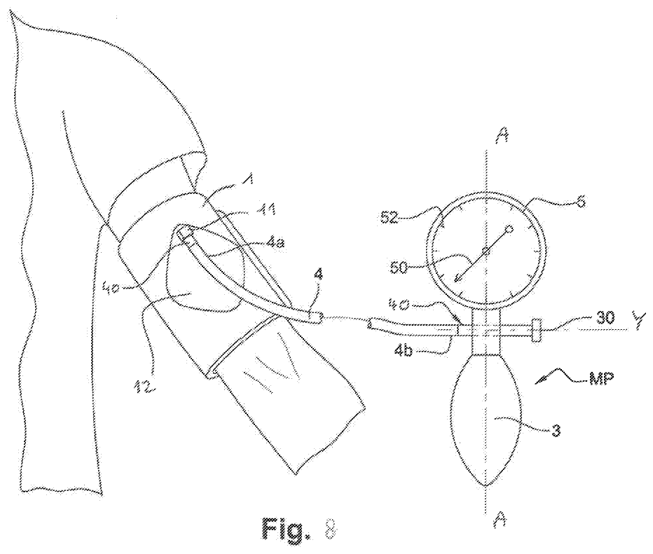

In FIG. 7, the tube 4 includes a male end-piece 41 at each end 4a, 4b. These male end-pieces 41 are suitable for being inserted into the complementary female connection holes 60 described hereinbelow in the description and which are located on the inflation bulb MP and optionally on the cuff 1.

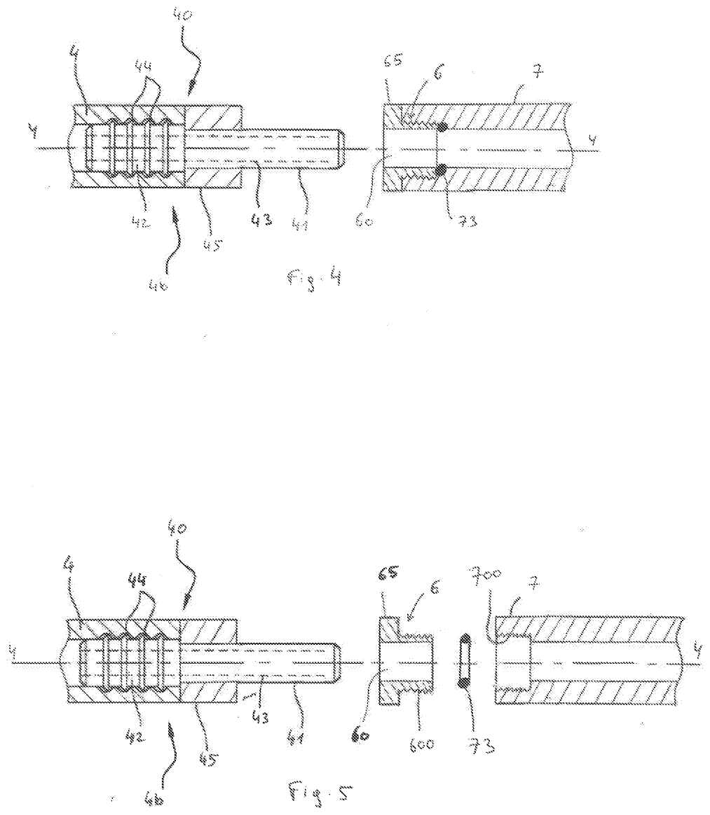

In reference more particularly to FIG. 4, the means of connection 40 is formed from the external male end-piece 41 and from an internal male end-piece 42, with these two end-pieces being connected by a portion 45, which preferably is in the form of a collar. The collar form facilitates in particular the manipulation of the means of connection 40 and simplifies the design thereof. In the rest of the description, the term “collar” is used as a synonym of “portion”.

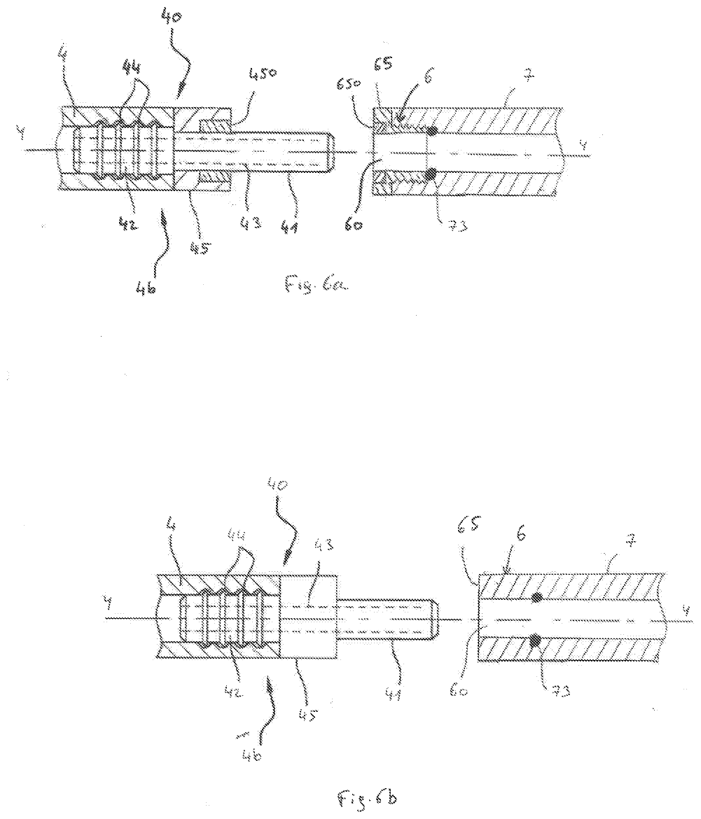

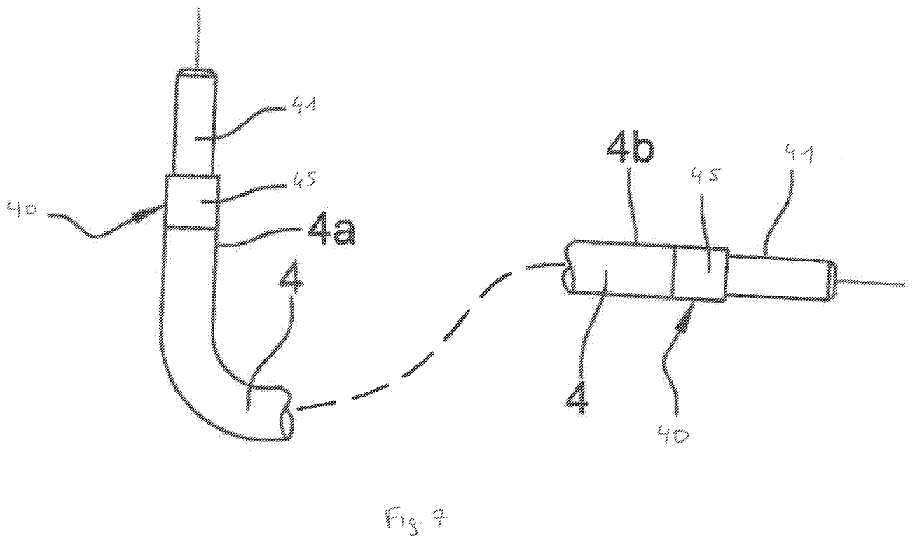

To further simplify the design thereof, this means of connection 40 can advantageously be manufactured in a single piece. In particular, the collar 45 is an integral part of the external male end-piece 41 (and where applicable of the internal end-piece 42) and forms a single-piece part with said external male end-piece (FIG. 6b).

The end-pieces 41, 42 and the collar 45 can be manufactured in a ferromagnetic material, such as a steel, or any other material that can be attracted by a magnet. In an embodiment alternative, the end-pieces 41, 42 and the collar 45 are manufactured in a magnetic material to form a permanent magnet. Preferably, a magnetic material from the following family is used: Neodymium-Iron-Boron, Samarium-Cobalt, alnicos, hard ferrite, magnetite alloy.

In FIGS. 4 and 5, the means of connection 40 is manufactured in two parts, the collar 45 being an added part and integral with the external male end-piece 41. This fastening is done for example by gluing, screwing or forced fitting. This solution has the advantage of being able to use two distinct materials: for example a plastic such as ABS for the end-pieces 41, 42 and a ferromagnetic material or a magnetic material for the collar 45. When the collar 45 is carried out in a magnetic material, it fully forms the permanent magnet.

In FIG. 6a, the collar 45 includes an arrangement wherein one or more ferromagnetic elements, or one or more permanent magnets 450 are installed. The collar 45 can in this case be made of plastic, such as ABS, for example.

The end-pieces 41, 42 and the collar 45 are axially pierced in such a way as to form a conduit 43 allowing for the circulation of air.

The external male end-piece 41 has, preferably, the form of a cylinder. This cylinder has for example an outer diameter comprised between 0.15 cm and 2 cm, and a length for example comprised between 0.5 cm and 2 cm. The outer wall of the end-piece 41 is smooth; regular, devoid of any air sealing element.

The internal male end-piece 42 has, preferably, the form of a cylinder similar to the one that form the external male element 41. This cylinder has for example a diameter comprised between 0.15 cm and 2 cm, and a length that can for example vary from 0.5 cm to 2 cm.

The internal male end-piece 42 is adapted to be inserted in a sealed manner into the respective end 4a, 4b of the flexible tube 4 in such a way that the means of connection 40 are integral with the latter. This internal male end-piece 42 includes at least one fastening collar 44 that cooperates with the inner wall of the tube 4. This fastening collar 44 makes it possible to insert it by force into the tube 4 and thus provide an airtight seal between the means of connection 40 and said tube. Advantageously, at least two collars 44, and preferably at least four fastening collars 44, are disposed around the cylinder of the end-piece 42. In practice, the outer diameter of the collars 44 is slightly greater than the inner diameter of the tube 4 (for example by 1 mm) in order to ensure the insertion by force of the internal male end-piece 42 and a plastic deformation of the inner wall of the tube 4, which deformation provides the airtight seal.

The collar 45 is disposed outside the tube 4 and bears against the respective end 4a, 4b of the latter. The collar 45 has for example the form of a cylinder of which the outer diameter corresponds to the outer diameter of the tube 4, its length being for example comprised between 0.5 cm and 1 cm.

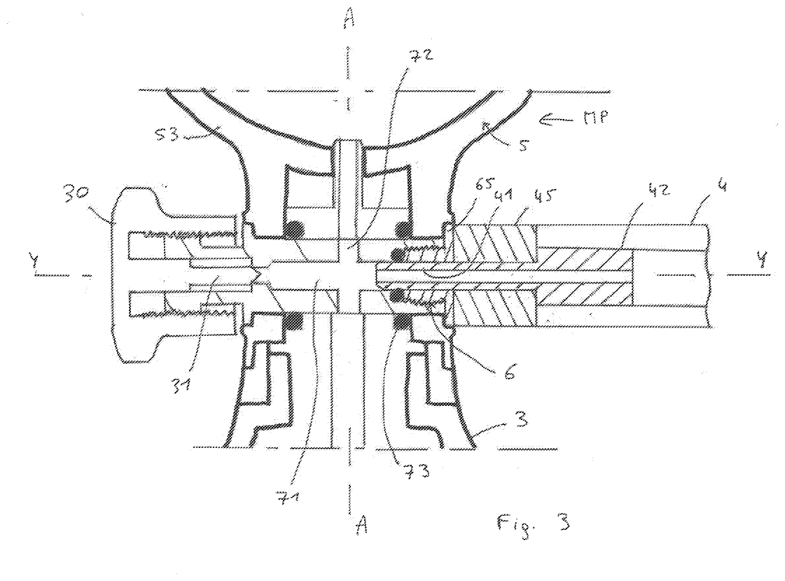

In reference to FIG. 3, a connection member 6 is mounted in the inflation bulb MP to connect the second end 4b of the tube 4 to said inflation bulb, such that said tube is adapted to provide a direct link between said inflation bulb and the cuff 1. The female hole 60 is made in this connection member 6 and is adapted to removably receive the male end-piece 41. As is shown in FIG. 3, the inlet of the female hole 60 is preferably arranged in the lateral edge of the inflation bulb MP, in such a way that its axis Y-Y is perpendicular to the longitudinal axis A-A of said inflation bulb. The applicant has observed that this orientation allows for an easier connection of the tube 4.

In FIG. 4, the female hole 60 has an inner cylindrical wall adapted to the structure of the external male element 41. More particularly, the cylinder of the external male end-piece 41 has an outer diameter adjusted to the inner diameter of the female hole 60. “Adjusted” means that the inner diameter of the female hole 60 is substantially equal to the outer diameter of the cylinder that forms the external male end-piece 41, and preferably slighter greater (for example by 0.5 mm) in order to prevent friction during the insertion of said end-piece 41. The female hole 60 has for example an inner diameter comprised between 0.15 cm and 2 cm, and a length corresponding substantially to that of the cylinder forming the external male end-piece 41, said male end-piece being however slightly longer in such a way that it extends beyond said female hole 60 (FIG. 3).

The connection member 6 comprises a portion 65 that cooperates with the complementary portion 45 provided on the male end-piece 71. The portion 65 advantageously is in the form of a collar.

As shown in FIG. 2, this collar 65 is located on the wall of the inflation bulb MP and is therefore perfectly accessible. The collar 65 has for example the form of a cylinder of which the outer diameter corresponds to the outer diameter of the collar 45, its length being for example comprised between 0.5 cm and 1 cm. When the male end-piece 41 is connected in the female hole 60, the collars 45, 65 are bearing against one another.

The collar 65 can be made from a ferromagnetic material or a magnetic material. When the collar 45 is made from a magnetic material, it fully forms a permanent magnet.

In FIG. 6a, the collar 65 includes an arrangement wherein one or more ferromagnetic elements, or one or more permanent magnets 650 are installed. The collar 65 can in this case be made from plastic, such as ABS, for example.

In order to provide an effective connection between the means of connection 40 of the tube 4 and the connection member 6, the portions 45 and 65 cooperate via magnetization. If the collar 45 includes a ferromagnetic material, the collar 65 includes a permanent magnet. If the collar 45 includes a permanent magnet, the collar 65 includes a ferromagnetic material. The collar 45 and the collar 65 can also both include a permanent magnet. In this latter case, the permanent magnets are arranged in such a way that their poles of opposite nature are facing each other and attract each other.

The collar 45 exerts an attractive force on the collar 65 (or inversely) which makes it possible to automatically insert the male element 41 into the female member 60, without additional force or intervention from the user. As soon as the male element 41 is engaged in the female member 60, the connection is established on its own, until the collar 45 abuts against the collar 65. When the collars 45, 65 are bearing one against the other, the magnetization force provides the maintaining in position of the male element 41 in the female member 60, thus making it possible to reduce the risks of cable disconnection 4 during an untimely pulling off. With a force of adhesion comprised between 1 kg and 3 kg (i.e. about 10-30 newtons) between the collars 45 and 65, a good compromise is obtained between: reduction in the risks of untimely disconnection of the cable 4 and easy voluntary disconnection of said cable.

In FIG. 3, a junction block 7 is installed outside the shell 53 of the inflation bulb MP, in an axial bore. This junction block 7 provides fluidic communication between the bulb 3, the pressure gauge 5, a valve 31 of the control device 30, and the connection member 6 of the tube 4. This junction block 7 is similar to the one described in patent document FR3051344 (SPENGLER) that those skilled in the art can refer to in case of need.

This block 7 has a general cylindrical outside form. It is advantageously made from a rigid material, preferably a plastic such as ABS, or a metal, such as a stainless steel. It is pierced axially in such a way as to form a channel 71 allowing the circulation of air. The block 7 has for example an inner diameter comprised between 0.2 cm and 1 cm and an outer diameter comprised between 0.4 cm and 1.5 cm. Its length is for example comprised between 1 cm and 3 cm.

The canal 71 has a first end at which is arranged the connection member 6, and a second end wherein the deflation valve 31 of the cuff 1 is engaged.

The block 7 also includes a bore 72 that extends along the vertical axis A-A of the inflation bulb MP and which is in fluidic communication with the channel 71. This bore 72 has a lower end in fluidic communication with the bulb 3 and an upper end in fluidic communication with the measuring chamber of the pressure gauge 5.

In FIGS. 3, 4, 5 and 6a, the connection member 6 is an added interface part, that is inserted into the first end of the block 7. To simplify the assembly thereof, this interface part 6 is preferably screwed into the first end of the block 7. Solutions by welding or forced fitting can however be considered, although these have the disadvantage of requiring additional tools and/or steps.

In a solution by screwing, the interface part 6 has an axially pierced cylindrical body and has an external thread 600 that engages in a complementary thread 700 made on the inner wall of the block 7, at the first end thereof. This threaded cylindrical body carries, at one of its ends, the collar 65. The interface part 6 is inserted into the block 7 until the rear face of the collar 65 abuts against the first end of the block 7.

FIG. 6b shows an embodiment alternative where the connection member 6 is an integral part of the block 7 and forms with the latter a single-piece part. The collar 65 is in this case an integral part of the block 7 located at the first end of the latter.

In FIGS. 3 to 5 and 6b, the block 7 is provided with at least one O-ring seal 73 that cooperates with the male end-piece 41 to provide an airtight seal between said male element and said junction block. This seal 73 is disposed in the axial bore of the block 7. It can be housed in a dedicated groove provided for this purpose in the inner wall of said bore, or be maintained in position by the interface part forming the connection member 6. The seal 73 is preferably made of rubber but it can also be made from another material, such as silicone. In an embodiment alternative shown in FIG. 6a, the seal 73 is directly integrated into the female connection hole 60 in order to provide an airtight seal between the male element 41 and said female hole.

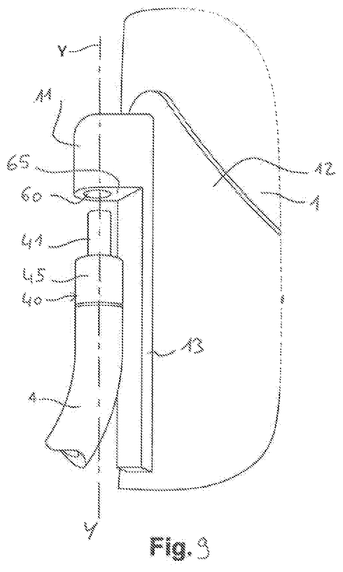

FIGS. 8 and 9 show the case where the first end 4a of the tube 4 is removably connected to the cuff 1, this tube providing a direct link between the inflation bulb MP and said cuff. In this case, the connection member 6 is mounted in the cuff 1, and more particularly arranged in a protuberance 11. Its female hole 60, in fluidic communication with the inflatable pouch of the cuff 1, is suitable for removably receiving the male end-piece 41 of the first end 4a.

The protuberance 11 is made projecting onto an interface part 12, which interface part acts as an interface between the inflatable pouch and the female hole 60 of the cuff 1. The protuberance 11 is advantageously made from a rigid material, plastic or metal. It could however be made from a semi-rigid or flexible plastic material.

The face of the protuberance 11 whereon the inlet of the female member 60 is arranged, corresponds to the portion 65 of the connection member 6 which cooperates via magnetization with the collar 45 of the male end-piece 41. Also, as described hereinabove, the portion 65 can include a permanent magnet in the case where the collar 45 is made from a ferromagnetic material or in the case where the collar 45 also includes a permanent magnet. The portion 65 can also be made from a ferromagnetic material in the case the collar 45 includes a permanent magnet.

An arrangement in relief 13, in the form of a shoe, is installed in front of the female hole 60 and in the axis Y-Y of the latter. This shoe 13 makes it possible to guide in position the male end-piece 41 of the tube 4. This shoe 13 also makes it possible to rigidify and to reinforce the interface piece 12. Its length is for example comprised between 1 cm and 5 cm, its width preferably corresponds to that of the protuberance 11, and its thickness is for example comprised between 2 mm and 1 cm. This shoe 13 can have a concavity in the axis Y in such a way as to facilitate the guiding of the end-piece 41.

The usage of the system object of the invention shall now be described in detail. The doctor has at his disposition: the cuff 1, a tube 4 and the inflation bulb MP.

When the doctor needs to use an inflation bulb, it is sufficient for him/her to place a cuff 1 of suitable size around the arm of the patient. He/she then connects one of the male end-pieces 41 of the tube 4 in the female connection hole 60 of the cuff 1 (FIG. 9) if the first end 4a of the latter is not already integral with said cuff (FIG. 1). The male end-piece 41 of the second end 4b of the tube 4 is connected in the female connection hole 60 of the inflation bulb MP of his choice, for example with a needle pressure gauge 5 and/or of a certain model or brand.

When the doctor needs to use another inflation bulb (for example with a digital display pressure gauge 5 and/or of another model or brand) it is sufficient for him/her to disconnect the male end-piece 41 from the second end 4b outside the female connection hole 60 of the inflation bulb MP. He/she then reconnects this male end-piece 41 in the female connection hole 60 of another inflation bulb MP.

The doctor can also easily use another cuff 1, while still retaining his inflation bulb MP.

If the first end 4a of the tube 4 is integral with the cuff 1 to be changed (configuration of FIG. 1), it is sufficient for him/her to disconnect the male end-piece 41 from the second end 4b outside the female connection hole 60 of the inflation bulb MP. He/she then reconnects the male end-piece 41 of the second end 4b of the tube 4 attached to another cuff 1, of a more suitable size, in the female connection hole 60 of the inflation bulb MP.

If the first end 4a of the tube 4 is removably connected to the cuff 1 to be changed (configuration in FIGS. 8 and 9), it is sufficient for him/her to disconnect the male end-piece 41 from the first end 4a outside the female connection hole 60 of said cuff. He/she then reconnects this male end-piece in the female connection hole 60 of another cuff 1 with a more suitable size. The male end-piece 41 of the second end 4b of the tube 4 remains connected in the female connection hole 60 of the inflation bulb MP.

The arrangement of the various elements and/or means and/or steps of the invention, in the embodiments described hereinabove, must not be understood as requiring such an arrangement in all the implementations. In any case, it will be understood that various modifications can be made to these elements and/or means and/or steps, without leaving the ideas and the scope of the invention. In particular:

The cuff 1 can be installed around a suitable member of the patient such as: arm, wrist, leg, etc.

Inflating means 3 that are more complex than a bulb can be used, such as a piston pump or a gear pump.

The internal end-piece 42 and the external end-piece 41 can be manufactured separately and assembled together later by screwing or thanks to a method of welding or gluing for example.

The section of the cylinder that comprises the external male end-piece 41 is not necessarily circular, it can for example be square, rectangular or elliptical.

The cuff 1 does not necessarily include an interface part 12. The female hole 60 can, for example, be arranged directly in the inflatable pouch. The protuberance 11 can also be directly attached to the inflatable pouch.

1-15. (canceled)

16. A connection system for a sphygmomanometer comprising:
an inflatable cuff adapted to be placed on a limb of a patient to be examined and/or monitored, the cuff including an inflatable pouch,
a flexible tube including a first end and a second end, the first end in fluid communication with the inflatable pouch of the cuff,
an inflation bulb formed by an inflator bulb connected to a pressure gauge for measuring blood pressure,
a connection member mounted in the inflation bulb to connect the second end of the tube to said inflation bulb such that said tube is adapted to provide a direct link between said inflation bulb and the cuff,
the second end of the tube includes a male end-piece and the connection member includes a female connection hole for removably receiving said male end-piece,
wherein:
the connection member comprises a portion that cooperates magnetically with a complementary portion provided on the male end-piece of the tube, and
at least one of the portions includes a permanent magnet providing magnetization.

17. The system according to claim 16, wherein:
a junction block is mounted in the inflation bulb, and the junction block is adapted to provide a fluidic communication between the inflator bulb, the pressure gauge, a device for controlling a valve, and the connection member, and
the connection member is an added interface part inserted into an end of the junction block.

18. The system according to claim 16, wherein:
a junction block is mounted in the inflation bulb, and the junction block is adapted to provide a fluidic communication between the inflator bulb, the pressure gauge, a device for controlling a valve, and the connection member, and
the connection member is an integral part of the junction block and forms with the latter a single-piece part.

19. The system according to claim 16, wherein the portion of the connection member is in the form of a collar.

20. The system according to claim 16, wherein the complementary portion provided on the male end-piece of the tube is in the form of a collar, and the collar is disposed outside of said tube and bears against the second end of said tube.

21. The system according to claim 20 taken in combination, wherein the collar of the male end-piece bears against the collar of the connection member when said male end-piece is connected in the female hole of said connection member.

22. The system according to claim 16, wherein:
the portion of the connection member includes a permanent magnet,
the complementary portion provided on the male end-piece includes a ferromagnetic material.

23. The system according to claim 16, wherein:
the portion of the connection member includes a ferromagnetic material, and
the complementary portion provided on the male end-piece includes a permanent magnet.

24. The system according to claim 16, wherein the portion of the connection member and the complementary portion provided on the male end-piece each include a permanent magnet, said permanent magnets being arranged in such a way that their poles of opposite nature are facing each other and attract each other.

25. The system according to claim 16, wherein the complementary portion provided on the male end-piece is an added part integral with said male end-piece.

26. The system according to claim 16, wherein the complementary portion provided on the male end-piece is an integral part of said male end-piece and forms with the latter a single-piece part.

27. The system according to claim 16, wherein:
a junction block is mounted in the inflation bulb (MP), and the junction block is adapted to provide a fluidic communication between the inflator bulb, the pressure gauge, a device for controlling a valve, and the connection member, and
the junction block is provided with a seal that cooperates with the male end-piece of the tube to provide an airtight seal between said male element and said junction block.

28. The system according to claim 16, wherein the female connection hole of the connection member is provided with a seal that cooperates with the male end-piece of the tube to provide an airtight seal between said male element and said female hole.

29. The system according to claim 16, wherein the male end-piece of the tube has an outer wall devoid of an air sealing element.

30. The system according to claim 16, wherein:
a connection member is mounted in the cuff to connect the first end of the tube to said cuff,
the first end of the tube includes a male end-piece and the connection member of the cuff includes a female connection hole for removably receiving said male end-piece,
the connection member of the cuff comprises a portion that cooperates magnetically with a complementary portion provided on the male end-piece of the first end of the tube, and
at least one of said portions includes a permanent magnet providing magnetization.
